(19) United States
(12) Patent Application Publication
(10) Pub. No.: US 2025/0037878 A1
(43) Pub. Date: Jan. 30, 2025

(54) Method for Optimizing an Entrainment of a Circadian Cycle

(71) Applicant: Arcascope Inc., Arlington, VA (US)

(72) Inventors: Olivia Walch, Falls Church, VA (US); Eric Canton, Eau Claire, WI (US); Kevin Hannay, Comfort, TX (US)

(21) Appl. No.: 18/912,127

(22) Filed: Oct. 10, 2024

Related U.S. Application Data
(63) Continuation-in-part of application No. 17/614,985, filed on Nov. 29, 2021.

(51) Int. Cl. G16H 50/30; G06F 17/17

(52) U.S. Cl. CPC G16H 50/30; G06F 17/17

(57) ABSTRACT

A method for determining a zeitgeber, K(t), for use in a circadian entrainment system to entrain an individual's circadian clock from a current circadian trajectory, x(t), towards a target circadian trajectory, y(t), can reduce a cost function C=C(x(t), y(t), K(t)) given an initial circadian state, x₀, and y(t), by (a) providing an initial zeitgeber, K₀(t), setting K(t)=K₀(t), (b) simulating x(t) in response to K₀(t), (c) computing a global cost function, C(x(t), y(t), K₀(t)), based on the simulation, (d) determining whether the global cost function meets a set of convergence criteria, and if not, (e) updating K(t) based on the global cost function, (f) updating the global cost function based on the updated zeitgeber K(t), (g) iterating until the global cost function meets the convergence criteria, and (h) providing the updated zeitgeber K(t) for use in the circadian entrainment system to entrain the individual's circadian clock.

### CROSS-REFERENCES TO PRIORITY AND RELATED APPLICATIONS

This application is a continuation-in-part of U.S. patent application Ser. No. 17/614,985, filed Nov. 29, 2021, and entitled “Method to Minimize the Cost of Entraining a Target Limit Cycle”, which in turn claims priority from PCT Application PCT/US2021/035024 filed May 28, 2021.

The entire disclosures of applications/patents recited above are hereby incorporated by reference, as if set forth in full in this document, for all purposes.

### FIELD

The present disclosure generally relates to circadian cycles and states and more particularly to methods and apparatus for optimizing an entrainment of a circadian cycle to a target cycle.

### STATEMENT REGARDING FEDERALLY SPONSORED RESEARCH OR DEVELOPMENT

This invention was made with government support under award number 2R44CA236557-02A1 awarded by the National Cancer Institute. The government has certain rights in the invention.

### BACKGROUND

Circadian rhythms are endogenous rhythms with a periodicity of approximately 24 hours. This endogenous rhythm regulates human sleep and wake cycles, hormonal activities, body temperature, and hunger and digestion. However, external causes may prevent a person from operating with his or her preferred circadian rhythm. These external causes include shift work, travel to a new time zone, staying up late on the weekend, or any deviation from a fixed daily schedule. Ideally, a person's circadian rhythm will quickly correct in response to external factors; i.e., by returning to a baseline after an acute disturbance, such as a late night, or by adjusting (entraining) to a new time zone. However, the larger the time change, the more difficult it will be for an individual to entrain to a new circadian rhythm. Moreover, if the person is on a very irregular schedule, such as shift work, he or she may never entrain, and instead will be in a state of perpetual misalignment from his or her preferred rhythm.

An individual may receive a prescription on how to entrain to a new circadian rhythm, or control a rhythm that is not entrained to achieve some other effect, such as maximizing sleep duration or minimizing the number of hours where one is fatigued on a work shift. Such a prescription considers zeitgebers or “time-givers.” Zeitgebers are inputs, such as light, darkness, exercise, amongst others, that influence an individual's circadian clock. These inputs can affect both the central circadian clock, the suprachiasmatic nucleus (SCN), as well as peripheral circadian oscillators, such as clocks in the stomach, liver, etc. Light is the most important input to the central clock, but exercise and hormones, like melatonin, also have an effect. Food is a zeitgeber for the clocks in peripheral organs. Caffeine has also been reported to be a zeitgeber, particularly in the evening.

Different methods of entraining to a new circadian rhythm might have disadvantages such as taking too long, requiring too much burden or expense, involving too much resource consumption, etc., and thus it would be useful to have methods an apparatus that optimize an entrainment system and/or method.

### SUMMARY

Methods and apparatus are described for determining a zeitgeber, K(t), for use in a circadian entrainment system to entrain an individual's circadian clock from a current circadian trajectory, x(t), to or towards a target circadian trajectory, y(t), that minimizes or reduces a cost function C=C(x(t), y(t), K(t)). This might comprise (a) identifying an initial circadian state, x₀, (b) identifying the target circadian trajectory y(t), (c) providing an initial zeitgeber, K₀(t), and setting the zeitgeber K(t)=K₀(t), (d) simulating the current circadian trajectory x(t) in response to the initial zeitgeber K₀(t), (e) computing a global cost function, C(x(t), y(t), K₀(t)), based on the simulation, (f) determining whether the global cost function meets a set of convergence criteria, (g) if the global cost function does not meet the set of convergence criteria, updating the zeitgeber K(t) based on the global cost function, (h) updating the global cost function based on the updated zeitgeber K(t), (i) iterating steps (g) and (h) until the global cost function meets the set of convergence criteria, and (j) providing the updated zeitgeber K(t) as the zeitgeber for use in the circadian entrainment system to entrain the individual's circadian clock.

Providing the initial zeitgeber, K₀(t), might comprise providing a zeitgeber for a point in time or providing a zeitgeber for a time range. The zeitgeber for use in the circadian entrainment system might comprise one or more of a duration of light, a duration of dark, and/or a timing of a zeitgeber event.

Simulating the current circadian trajectory x(t) in response to the initial zeitgeber K₀(t) might comprise: (a) receiving at least one dataset, (b) developing at least one zeitgeber history, using the dataset, (c) providing the dataset to a network of coupled oscillators, represented as nodes connected by edges, over a time period [tᵢ, tⱼ], and (d) using an output of the network of coupled oscillators for determining the global cost function. The parameters of the coupled oscillators might be tuned using an autoencoder neural network. The network of coupled oscillators might be modeled as a reduction of a coupled phase oscillator network. The dataset might include data taken from one or more of an actigraphy dataset, a heart rate dataset, a light dataset, and/or a temperature dataset, and/or wherein the dataset is provided by a wearable device. A first x(t) derived from a first dataset and a second x(t) might be derived from a second dataset, with the first x(t) and the second x(t) weighted by confidence in a quality of data. The first dataset and the second dataset might be combined using a Kalman filter.

In simulating the current circadian trajectory x(t) in response to the initial zeitgeber K₀(t), wherein updating the zeitgeber K(t) based on the global cost function might comprise: providing a lower bound for K(t), providing an upper bound for K(t), sampling components of K(t) within the lower bound and the upper bound, to derive a set of sampled components, calculating the global cost function using the set of sampled components, the current circadian trajectory x(t), the target circadian trajectory y(t), and the zeitgeber K(t), interpolating a set of cost values using the sampled components to identify possible locations of minima of the global cost function, updating K(t) in identified minima locations of the possible locations at a denser resolution, and repeating the interpolating using the denser resolution. The lower bound and the upper bound might be set to be a minimum allowable time and a maximum allowable time, respectively, spent in either dark or light, with a first light duration, a first dark duration, a last light duration, and a last dark duration having no lower bounds, and wherein, for a specified activity, k, specified once every N hours, the lower bound is k*N and the upper bound is (k+1)*N. Sampling components of K(t) might be done using Latin hypercube sampling. The sampled cost values might be interpolated with polynomials, trigonometry functions, or neural networks.

Determining the zeitgeber might comprise: (a) calculating components of x₀, (b) sampling from a range of possible zeitgeber choices K⁽ⁱ⁾, K⁽²⁾, . . . K^{(N)}, where each zeitgeber choice represents a different choice of stimulus for a single time step, (c) simulating x(t) over a single time step to get x₁ for each zeitgeber choice K⁽ⁱ⁾, (d) calculating a cost for all zeitgeber choices K⁽ⁱ⁾, (e) choosing the zeitgeber choice with a lowest cost, Cⁱ, as a chosen zeitgeber, (f) setting K(t₀) equal to the chosen zeitgeber, and (g) repeating steps (c) through (f) for xₙ.

In some embodiments, C(x(t), y(t), K(t))=XC_{A}+YC_{B}+ . . . +ZC_{N}, with C_{A}, C_{B}, . . . , C_{N} each representing different possible criteria of interest and X, Y, Z each representing weightings of these costs. C_{A}, C_{B}, . . . , C_{N} might be considered according to a defined hierarchy in evaluating a zeitgeber. Each of C_{A}, C_{B}, . . . , C_{N} might be based on one or more of a phase cost, a feasibility cost, a sleep duration, an alertness value, and/or a social jetlag cost.

The global cost function representing a cost to approximately entrain to a target circadian state might be minimized when C(x(t), y(t), K(t)) is less than a threshold value of cost. In some embodiments, C(x(t), y(t), K(t))=C_{A}*C_{B}*. . . *C_{N}, with each C_{A}, C_{B}, . . . , C_{N} having a minimum and maximum value.

The global cost function C might be based on one or more of a phase cost, a feasibility cost, a sleep duration, an alertness value, and/or a social jetlag cost.

A computer system might be provided for generating a zeitgeber, K(t), for use in a circadian entrainment system to entrain an individual's circadian clock from a current circadian trajectory, x(t), to or towards a target circadian trajectory, y(t), that minimizes or reduces a cost function C=C(x(t), y(t), K(t)), comprising at least one processor, and a computer-readable medium storing instructions. The instructions might be to (a) identify an initial circadian state, x₀, (b) identify the target circadian trajectory y(t), (c) provide an initial zeitgeber, K₀(t), and setting the zeitgeber K(t)=K₀(t), (d) simulate the current circadian trajectory x(t) in response to the initial zeitgeber K₀(t), (e) compute a global cost function, C(x(t), y(t), K₀(t)), based on the simulation, (f) determine whether the global cost function meets a set of convergence criteria, (g) test whether the global cost function meets the set of convergence criteria, (h) update the zeitgeber K(t) based on the global cost function upon determining that the global cost function does not meet the set of convergence criteria, (i) update the global cost function based on the updated zeitgeber K(t), (j) iterating steps (h) and (i) until the global cost function meets the set of convergence criteria, and (k) providing the updated zeitgeber K(t) as the zeitgeber for use in the circadian entrainment system to entrain the individual's circadian clock.

The computer system might further comprise a network of coupled oscillators represented as nodes connected by edges, wherein the computer-readable medium storing instructions further stores instructions to (l) receive at least one dataset, (m) develop at least one zeitgeber history, using the dataset, (n) provide the dataset to the network of coupled oscillators over a time period [tᵢ, tⱼ], and (o) use an output of the network of coupled oscillators for determining the global cost function.

The computer system might further comprise computer-readable medium storing instructions to (l) provide a lower bound for K(t) and an upper bound for K(t), (m) sample components of K(t) within the lower bound and the upper bound, to derive a set of sampled components, (n) calculate the global cost function using the set of sampled components, the current circadian trajectory x(t), the target circadian trajectory y(t), and the zeitgeber K(t), (o) interpolate a set of cost values using the sampled components to identify possible locations of minima of the global cost function, (p) update K(t) in identified minima locations of the possible locations at a denser resolution, and (q) repeating the interpolating using the denser resolution.

The computer system might further comprise computer-readable medium storing instructions to (l) calculating components of x₀, (m) sampling from a range of possible zeitgeber choices K⁽¹⁾, K⁽²⁾, . . . K^{(N)}, where each zeitgeber choice K⁽ⁱ⁾ represents a different choice of stimulus for a single time step, (n) simulating x(t) over a single time step to get x₁ for each zeitgeber choice K⁽ⁱ⁾. (o) calculating a cost for all zeitgeber choices K⁽ⁱ⁾, (p) choosing the zeitgeber choice K⁽ⁱ⁾ with a lowest cost, Cⁱ, as a chosen zeitgeber, (q) setting K(t₀) equal to the chosen zeitgeber, and (r) repeating steps (n) through (q) for xₙ.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of methods and apparatus, as defined in the claims, is provided in the following written description of various embodiments of the disclosure and illustrated in the accompanying drawings.

### BRIEF DESCRIPTION OF THE DRAWINGS

The following detailed description will be better understood when read in conjunction with the appended drawings, in which there is shown example embodiments for the purposes of illustration. It should be understood, however, that the present disclosure is not limited to the precise arrangements and instrumentalities shown. In the drawings:

FIG. 1 is a graphical representation of a circadian rhythm and a zeitgeber.

FIG. 2 shows graphical representations of a circadian rhythm.

FIG. 3 is a flowchart of a method for optimizing entrainment to a target circadian state, taking into account a cost function representing a cost of entrainment.

FIG. 4 is a flowchart of a method for simulating and/or determining an estimated circadian trajectory, x(t), of an individual using a network of coupled oscillators.

FIG. 5 is a flowchart of a method for simulating and/or determining an estimated circadian trajectory, x(t), of an individual using preprocessing.

FIG. 6 is a flowchart of a method for simulating and/or determining an estimated circadian trajectory, x(t), of an individual using compositing of a plurality of time series.

FIG. 7 is a flowchart of a method for computing a stimulus time series, K(t), as might be used to entrain a circadian trajectory.

FIG. 8 illustrates a cost function value generator along with examples of input cost components and a cost output.

FIG. 9 is a flowchart of a method for computing a stimulus time series, K(t), with constraints.

FIG. 10 is a flowchart of a method for computing a stimulus time series of input vectors, \vec{K}(t).

FIG. 11 illustrates an example computer system memory structure as might be used in performing methods described herein, according to various embodiments.

FIG. 12 is a block diagram illustrating an example computer system upon which the systems and methods illustrated herein and in FIG. 11 may be implemented, according to various embodiments.

### DETAILED DESCRIPTION

In the following detailed description, reference is made to the accompanying drawings, which form a part hereof. In the drawings, the use of similar or the same symbol in different drawings typically indicates similar or identical items, unless context dictates otherwise.

The illustrative embodiments described in the detailed description, drawings, and claims are not meant to be limiting. Other embodiments may be utilized, and other changes may be made, without departing from the spirit or scope of the subject matter presented here.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken as limiting.

The present application uses formal outline headings for clarity of presentation. However, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, the use of the formal outline headings is not intended to be in any way limiting.

As explained herein, a circadian entrainment system is described. A circadian entrainment system might take in environmental inputs, user inputs, and/or other data and might provide outputs to be used by a user, a wearable device, or other equipment where the outputs are useful for entraining a user's circadian trajectory towards a target circadian trajectory. A circadian trajectory generally corresponds to a representation over time of a person's circadian rhythm at times along the time span represented by the circadian trajectory. It should be noted that the apparatus and methods described herein could be used for entraining nonhuman circadian trajectories as well. Entraining a circadian trajectory can be used to change a person's circadian states at particular times of the day from their current states to a set of desired states. This might be useful, for example, in changing work schedules, addressing jet lag, etc.

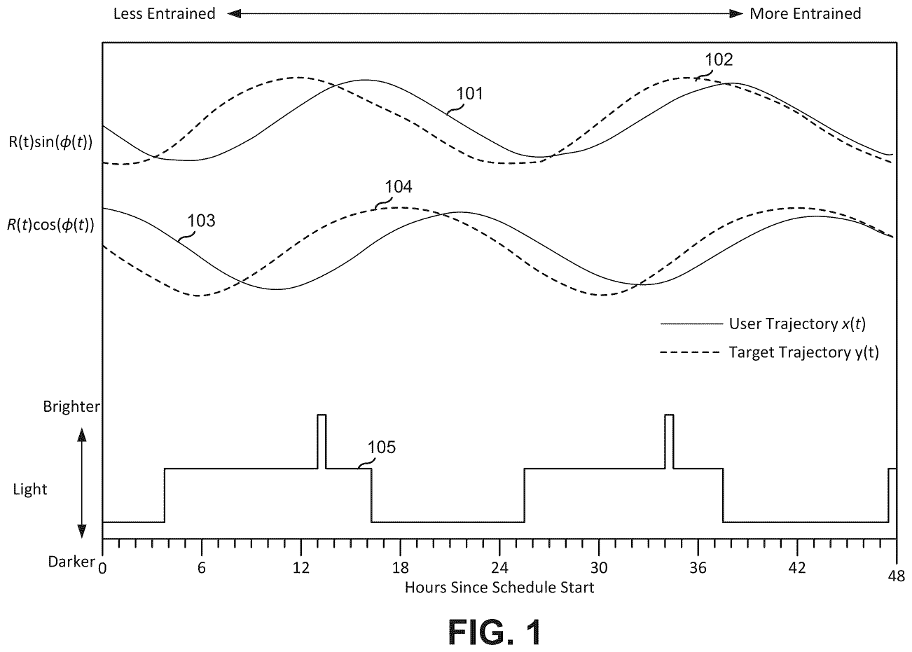

FIG. 1 illustrates an example of circadian trajectories and entrainment, showing a graphical representation of a circadian rhythm as it is entrained and a zeitgeber. In this example, circadian trajectories have periods of around 24 hours and can be represented by curves as shown in FIG. 1. In FIG. 1, solid lines 101, 103 represent a circadian trajectory starting from an initial circadian state as might represent a progression of a user's circadian state over the course of a few days in the absence of any intervention and dashed lines 102, 104 represent a target circadian trajectory. A circadian trajectory can be represented by a complex value over a period of time. In the examples shown, the circadian trajectory might be some circadian state value corresponding to a circadian level over time.

In depicting such circadian trajectories in FIG. 1, their real and imaginary components are presented, wherein R(t)sin(ϕ(t)) (lines 101, 102) represents the real component of the circadian trajectory over time and R(t)cos(ϕ(t)) (lines 103, 104) represents the imaginary component of the circadian trajectory over time. While R(t)sin(ϕ(t)) and R(t)cos(ϕ(t)) might appear as sinusoids in FIG. 1, that need not be the case.

In some cases, a circadian state might be represented by more than just one complex number. For example, circadian state might include an SCN state, a “light responsiveness” state, etc. A circadian entrainment system can provide outputs that direct inputs to the user, such as lighting levels, user prompts (e.g., to exercise, to intake melatonin, food, caffeine, etc.), and/or other output signals, that can cause a shifting of the user's circadian state over time to more align, or entrain, toward the target circadian trajectory. A circadian entrainment system might be implemented in hardware, with a processor, in software to be executed by a processor, firmware, or some combination. The circadian entrainment system might be software running on a wearable device and might communicate with an environmental management system, such as a home lighting control system, HVAC system, etc.

Some of the circadian entrainment system outputs can relate to zeitgebers that influence an individual's one or more internal circadian clocks. Zeitgebers can affect the individual's central circadian clock, the individual's suprachiasmatic nucleus (SCN), and the individual's peripheral circadian oscillators, such as clocks in the stomach, liver, etc. One or more zeitgebers might be specified to entrain the patient's starting circadian state to align the patient's circadian cycle with a target circadian cycle. One or more zeitgebers might be provided to the patient as a prescription.

A zeitgeber might be represented by a curve, such as zeitgeber curve 105 shown in FIG. 1. In this example, the zeitgeber is an input to a light-providing system that provides the user with an environment over time that might vary between a bright environment and a dark environment in the pattern illustrated by the curve in FIG. 1. While the zeitgeber curve 105 is shown as a step function curve, zeitgeber curves need not be limited to step functions.

Various mathematical expressions are used herein and might represent quantities, functions, computations, etc. performed by the circadian entrainment system. As a convention herein, “t” represents an independent variable corresponding to time, “tᵢ” refers to a specific time t=tᵢ such as to referring to an initial or starting time, x(t) refers to a circadian trajectory of the individual, x₀ would or x(t₀) would refer to an initial circadian state of the individual at time to), and y(t) refers to a target circadian trajectory for the individual. In some cases when represented for computer manipulation, the circadian trajectory of the individual, x(t), is an extrapolation corresponding to a computed future circadian trajectory of the individual, such as a computed estimate for the individual from the current time through the following 48 hours.

A zeitgeber might be expressed as K(t) that is stored in computer memory as a lookup table, a function, a stimulus time series, etc. A zeitgeber K(t) might be used by a circadian entrainment system to entrain an individual's circadian clock from a current circadian trajectory x(t) to, or towards, a target circadian trajectory y(t). The current circadian trajectory x(t) might be computed from a stimulus history of inputs and outputs that can be used to track a historical circadian state and y(t) might be a stimulus prescription designed to shift the individual's clock(s) prospectively in the future. K₀ or K(t₀) might refer to an initial zeitgeber value.

The circadian entrainment system might select or compute a zeitgeber K(t) that entrains an individual's circadian clock from a current circadian trajectory x(t) to, or towards, a target circadian trajectory y(t) wherein that selection or computation takes into account a cost function value. For example, in selecting between two zeitgebers K₁ and K₂ that could get the individual's circadian clock from x(t) to y(t), the circadian entrainment system might compute a cost function, C, for each of the zeitgebers (e.g., C₁ and C₂) and select the lesser cost of C₁(t) and C₂(t). A cost function might be expressed as C(x(t), y(t), K(t)), indicating that the cost can depend on the individual's actual, simulated, or predicted circadian trajectory, the target circadian trajectory, and the zeitgeber being used.

The cost function can represent a degree of burden, expense, resource consumption, etc. that would result from the use of a given zeitgeber. The cost function might be representative of some measurable quantities and/or some subjective quantities that can be represented in electronic or data form. For example, one cost component might be the number of hours of what would otherwise be productive hours that are unproductive while the circadian trajectory is being entrained towards a target circadian trajectory, which could be measured as a quantity. Another example might be a measure of subjective user preferences.

For example, an individual who really dislikes having to wake up early could provide data to be used as a component of cost with that component being higher where entrainment requires waking up early. In such a case, a circadian entrainment system might determine that adjusting lighting or exciting the individual at certain times of the prior day would be as effective in shifting their trajectory as getting them to wake up early and thus would have similar costs to achieve, but when taking into account the subjective cost component expressed by the individual, would find that the former approach has a lower computed cost.

In another example, the individual might have reasons to want to adjust as quickly as possible, in which case a circadian entrainment system knowing that subjective preference might assign a cost component that is a monotonic function of how long the user will spend in an adjusting-but-not-yet-adjusted state.

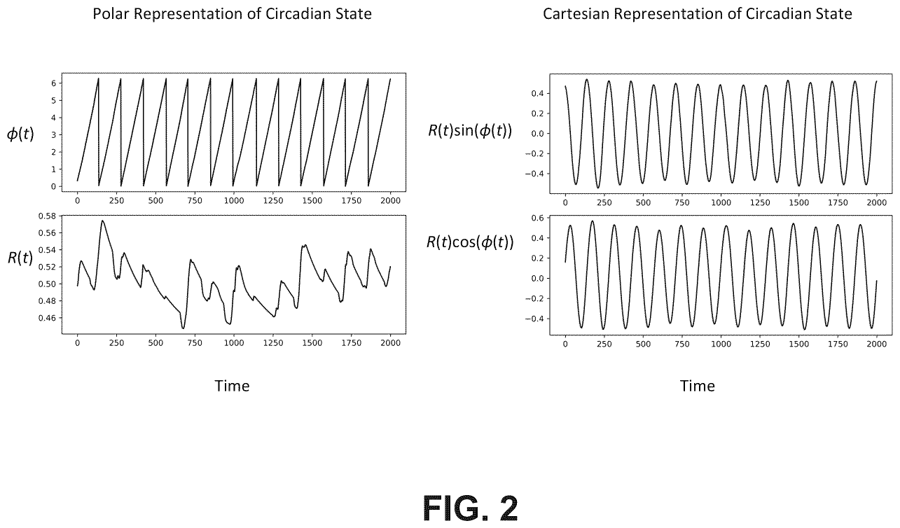

FIG. 2 shows graphical representations of a circadian rhythm. The circadian rhythm might be represented by a complex value function that varies over time and has an approximate periodicity, which might be more or less 24 hours, where the value of the complex value at a given time corresponds to a circadian state at that given time. The complex value function, expressed as polar components R(t) and ϕ(t), is shown on the left side of FIG. 2. As shown in the upper left, the phase ϕ(t) ramps up and might be modulo some value, shown as resetting each cycle. The y-axis might be normalized so that the phase varies within some range of values, in this specific example being from around 0 to around 2*π. The amplitude of the complex value function, R(t), in the lower left shows variations over the same time scale as in the upper left, with the value varying over time within the range of values shown.

A circadian rhythm, or the complex value function representing a circadian trajectory might be stored in computer-readable memory as complex value samples. In a specific example, the samples might each represent a value of circadian state at 10-minute intervals. In FIG. 2, with the units being the number of 10-minute intervals, 2000 of such intervals span 2000*10/(24*60), or about 14 days. In FIG. 2, the phase might range from 0 to around 2*71 and the amplitude of the complex value function R(t) might range from 0.45 to 0.58, representing some measurable aspect of a person's circadian rhythm, such as a suprachiasmatic nucleus (SCN), a peripheral circadian oscillator, an internal clock, or other circadian effects described herein or elsewhere.

On the right side of FIG. 2, corresponding Cartesian presentations of the complex value for the circadian state are plotted. In the upper right, R(t)sin(ϕ(t)), the imaginary component of the complex value is plotted, while in the lower right, R(t)cos(ϕ(t)), the real component of the complex value is plotted. In some cases, illustrating a circadian trajectory in polar components might more illuminate variations over time than would be apparent from illustrations of the real and imaginary components.

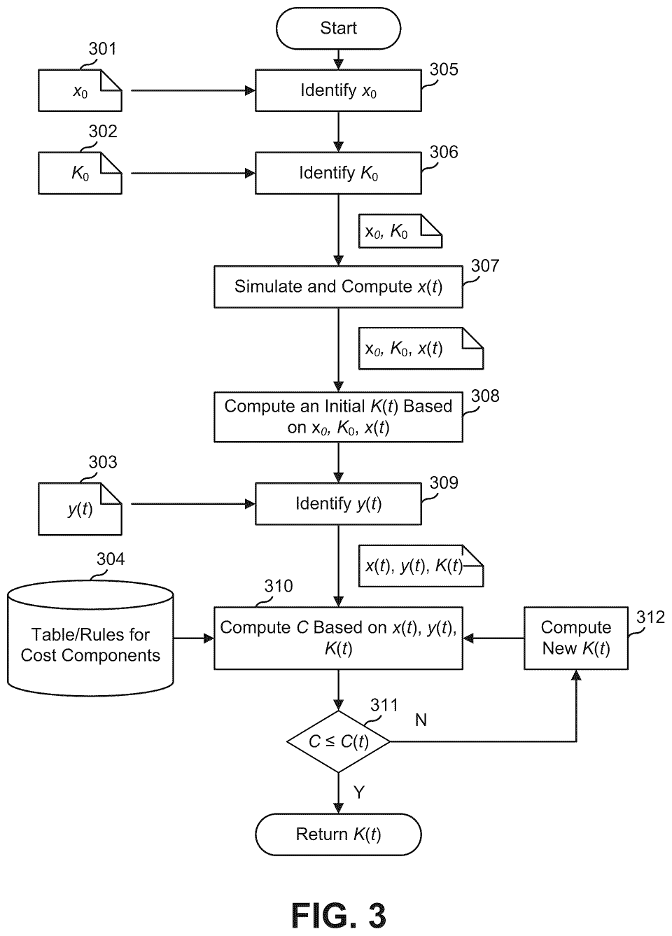

FIG. 3 is a flowchart of a method for optimizing entrainment to a target circadian state, taking into account a cost function representing a cost of entrainment. The method might be used by a circadian entrainment system to find a zeitgeber that provides a minimal cost value for an entrainment. As shown in FIG. 3, the method might comprise identifying (step 305) an initial circadian state, x₀ (element 301), of an individual, identifying (step 309) a target circadian trajectory y(t) (element 303), and prescribing (step 306) an initial zeitgeber, K₀ (element 302). Some of these steps can be done in an order other than that shown in FIG. 3. A circadian entrainment system might perform a method of simulating, determining, and/or computing (step 308) an initial zeitgeber, K(t) from x₀, K₀, and x(t), simulating (step 307) the individual's circadian trajectory, x(t), over time in response to K₀, and computing (step 310) a cost function value, C, over a time range spanned by K(t) based on x(t), y(t), K(t), and/or a set of cost component rules (304). An optimal zeitgeber might be found based on when C is computed or determined to be less than a threshold cost function value, C_{T} (step 311) and a new K(t) computed (step 312) as needed.

The zeitgebers, such as K₀ and K(t), might be specified as a series of values or a continuous function, with point values being specified for a particular time or a specific time range. The steps shown in FIG. 3 might be performed by a computer appropriately programmed and provided access to electronically readable representations of x₀ (element 301), initial zeitgeber, K₀ (element 302), target circadian trajectory y(t) (element 303), and set of cost component rules (304).

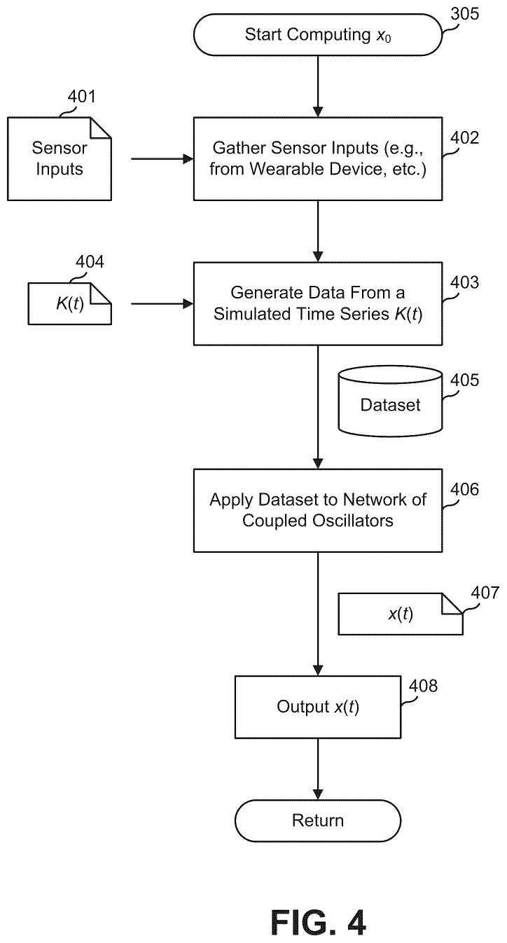

FIG. 4 is a flowchart of a method for simulating and/or determining an estimated circadian trajectory, x(t), of an individual using a network of coupled oscillators in response to a prescribed stimulus history K(t) (404), either at time t or over a time range [tᵢ, tⱼ]. The method might comprise gathering (step 402) sensor inputs (element 401), generating (step 403) at least one dataset (step 402) related to K(t) (404), applying (step 406) the dataset (element 405) to a network of nonlinear coupled oscillators represented by nodes connected by edges, over a period of time, to compute a representation (element 407) of x(t) and outputting (step 408) that representation.

In one embodiment, the network of coupled oscillators is modeled as modified phase oscillators. In another embodiment, the network of coupled oscillators is modeled as Kuramoto oscillators. In one embodiment, the stimulus time series might be a dataset provided by a wearable device that collects data on the individual's heart rate, activity, light exposure, sleep cycle, amongst others. In one embodiment, the stimulus time series is a prescribed pattern of zeitgebers that has been selected to phase shift the clock, and not drawn from wearables.

The network of coupled oscillators can simulate neurons in the brain's SCN in a way that parallels how light input is received by the brain (e.g., brighter light is a stronger stimulus, which has more action potential firing in a neuron). The coupling of these oscillators allows for the extraction of a “collective phase” and “collective amplitude” which together can comprise a representation of circadian state.

In one embodiment, the dataset may be activity or actigraphy data. In one embodiment, the dataset may be heart rate data. In one embodiment, the dataset may be light data. In one embodiment, the dataset may comprise data that is actigraphy data, heart rate data and light data. In one embodiment, actigraphy data is normalized between 0 and 1.

In an embodiment, actigraphy data is uncorrelated from heart rate data to account for the correlation between heart rate and activity to create a heart rate dataset with the effects of activity removed. The corrected heart rate set is normalized between 0 and 1. In an embodiment, light data is normalized between 0 and 1. In cases where the dataset provides data for continuous light, the light dataset might be weighted for the earliest light to be most important.

The steps shown in FIG. 4 might be performed by a computer appropriately programmed and provided access to electronically readable representations of sensor inputs (element 401) and of K(t) (404), and having memory or other storage for a dataset (element 405), and the representation (element 407) of x(t).

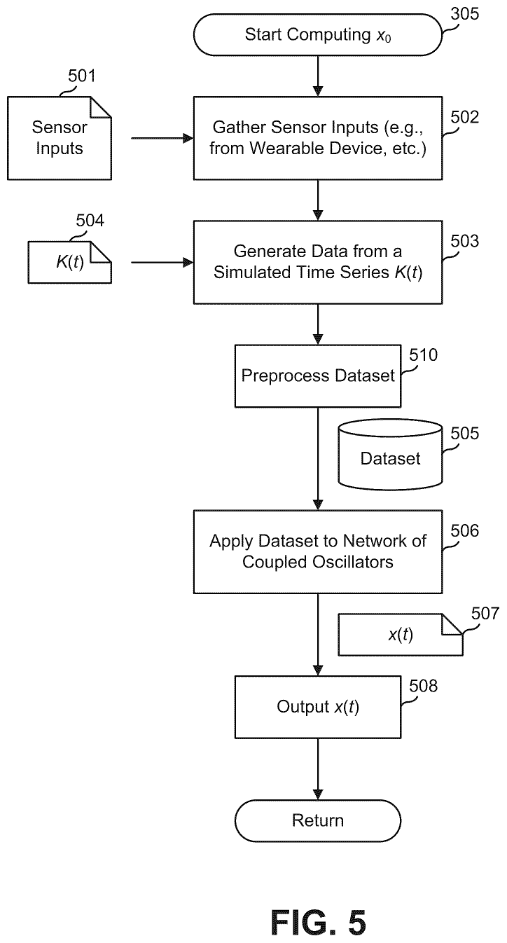

FIG. 5 is a flowchart of a method for simulating and/or determining an estimated circadian trajectory, x(t), of an individual using preprocessing. This is similar to the method shown in FIG. 4, with steps 502, 503, 506, and 508 being similar to steps shown in FIG. 4 and elements 501, 504, and 505 being similar to elements shown in FIG. 4. In this method, dataset 505 is pre-processed (step 510) before it is provided to the network of coupled oscillators. In one embodiment, pre-processing is normalization. The steps shown in FIG. 5 might be performed by a computer appropriately programmed and provided access to electronically readable representations, as with those of FIG. 4.

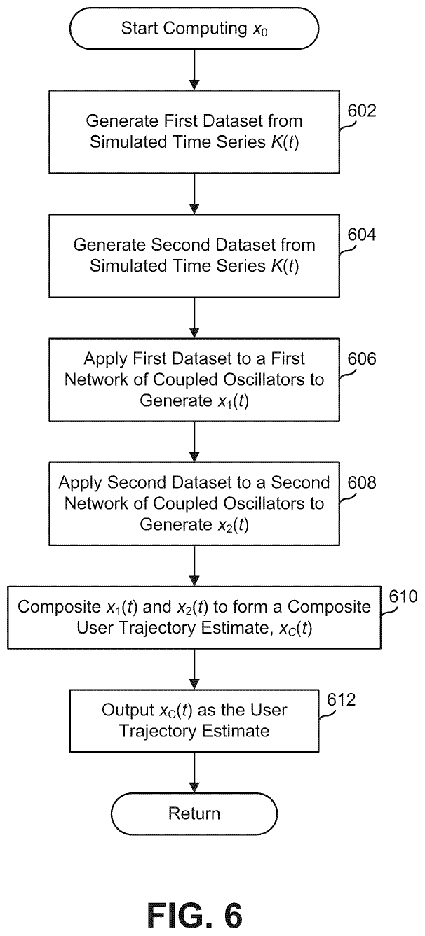

FIG. 6 is a flowchart of a method for simulating and/or determining an estimated circadian trajectory, x(t), of an individual using compositing of a plurality of time series. As illustrated there, multiple datasets might be received. As illustrated in steps 602, 604, 606, 608, 610, and 612, two datasets are composited, but it should be understood that more than two datasets could be composited. Where confidence in at least one dataset is greater than the confidence in at least one other dataset, the compositing could weight or discount datasets. Circadian trajectories could be determined, perhaps one per dataset with those having a higher confidence being up-weighted. For instance, given x_{A}(t) arrived at using a heart rate stimulus time series and x_{B}(t) arrived at using a temperature stimulus time series, and assuming that there is missing data from hours 20 to 30 for x_{A}(t) but not x_{B}(t), x_{B}(t) would be prioritized during the period of time when x_{A}(t) is missing data. In an embodiment, the filtering method used for up-weighting and down-weighting datasets having varying confidences is through the use of a Kalman filter.

The steps shown in FIG. 6 might be performed by a computer appropriately programmed and provided access to electronically readable representations of input values.

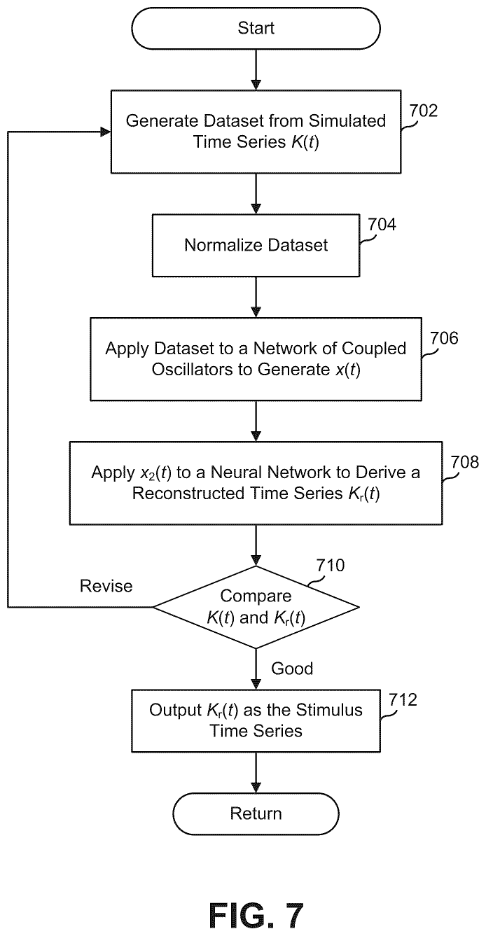

FIG. 7 is a flowchart of a method for computing a stimulus time series, K(t), as might be used to entrain a circadian trajectory. Parameters of a network of coupled nonlinear oscillators, such as their coupling strength, might be tuned using an autoencoder neural net. In particular, an output x(t) might be provided as an input to a neural net and used to construct a stimulus history K(t), arriving at an approximation Kᵣ(t). The parameters in the network of coupled nonlinear oscillators might then be updated to reduce the difference between K(t) and Kᵣ(t). As shown in FIG. 7, an example method might involve generating a dataset from simulated time series K(t) (step 702), normalizing the dataset (step 704), applying the dataset to a network of coupled oscillators to generate a simulated individual circadian trajectory, x(t) (step 706), applying x(t) to a neural network to derive a reconstructed time series Kᵣ(t) (step 708), and comparing K(t) and Kᵣ(t) (step 710). If the difference between K(t) and Kᵣ(t) is the small enough, the method would output Kᵣ(t) as the stimulus time series (step 712). If not, the method would flow back to step 702 to generate a new dataset using Kᵣ(t) as the simulated time series K(t).

In one embodiment, the network of coupled nonlinear oscillators is represented by a smaller set of equations which approximate its behavior, such as a dimensional reduction, or a type of limit cycle oscillator, such as a van der Pol oscillator. The steps shown in FIG. 7 might be performed by a computer appropriately programmed and provided access to electronically readable representations of the input and outputs shown, and the datasets referenced therein.

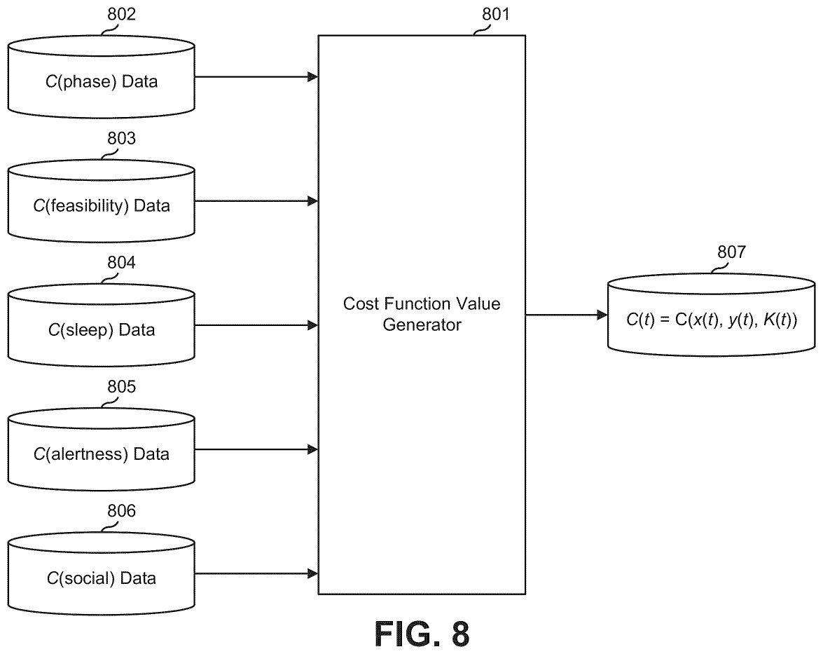

FIG. 8 illustrates a cost function value generator along with examples of input cost components and a cost output. Each of the cost components might be valued as of at time t or over a time range [tᵢ, tⱼ]. In one embodiment, cost might be represented by a phase cost value, Cₚₕₐₛₑ, from phase cost data 802. A phase cost value might be determined by the difference between a simulated trajectory x(t) and a target trajectory y(t). In an embodiment, cost might be represented by a feasibility cost value, C_{feasibility}, from feasibility cost data 803. A feasibility cost might be determined from a zeitgeber K(t) and might be a weighted sum of occurrences when bright light is recommended when the sunlight is unavailable and when darkness is recommended during sunlight hours.

In an embodiment, cost might be represented by a sleep duration cost value, Cₛₗₑₑₚ, from sleep cost data 804. Sleep duration cost might be determined by a simulated sleep model output, W(t), wherein W(t) represents sleep/wake status of an individual over the course of a trajectory, which might be determined using a zeitgeber schedule K(t) and a circadian trajectory x(t) as inputs into a sleep model. A sleep model might be implemented by a computer as a two-process model of sleep as explained below.

In a model, W(t) might represent a simulated pattern of sleep and wake as a function of time. In this model, W(tₖ)=0 if an individual is predicted, by a sleep model, to be asleep at time tₖ and W(tₖ)=1 if an individual is predicted, by the sleep model, to be awake at time tₖ. W(t) can be a function of x(t).

In an embodiment, cost might be represented by an alertness cost value, Cₐₗₑᵣₜₙₑₛₛ, from alertness cost data 805. Alertness cost might be determined by a simulated alertness function, A(t), which might be determined using a zeitgeber schedule K(t) and a circadian trajectory x(t) as inputs into a fatigue model that is representable in computer memory. This cost might reflect the number of hours an individual spends below an alertness threshold over the course of a day. Alertness can be calculated using a sleep model that can include caffeine. Certain hours (e.g., working hours) can be weighted as part of this cost.

A(t) might represent a simulated prediction of alertness as a function of time, wherein A(tₖ) is a high value if the person is predicted, by a fatigue model, to be alert at time tₖ and A(tₖ) is a low value if the person is predicted, by a sleep model or fatigue model, to be highly fatigued at time tₖ. A(t) can be a function of x(t). If F( ) is an embodiment of a fatigue model, A(t) might be computed as A(t)=F(x(t), K(t)).

In one embodiment, cost might be represented by a social jetlag cost value, C_{social}, from social cost data 806. Social cost data might be is determined using a simulated sleep model output W(t). This cost might reflect a difference between wake-up time and a desired wake-up on a prescribed light schedule such as the wake time predicted to correspond to a target limit cycle y(t).

In one embodiment, a cost function value generator 801 computes a cost function, C(t) 807 from one or more of phase cost data 802, feasibility cost data 803, sleep cost data 804, alertness cost data 805, and/or social cost data 806. Cost function value generator 801 might be implemented by a computer appropriately programmed and provided access to electronically readable representations of the data elements referenced therein.

In an embodiment, K(t) represents a pulse of an activity (e.g., complete 30 minutes of exercise, consume some melatonin, amongst others), where pulses can represent prescriptions for activities of any length over a time interval. In an embodiment, a zeitgeber is a period of light exposure and/or lack of light exposure. Light exposure may be light of varying brightness. For example, K(t) might be a prescription of low light for four hours and darkness for ten hours. In an embodiment, light having a defined quality can be prescribed over a period defined not to exceed an upper limit duration or drop below a lower duration, thus defining a light budget. For example, a zeitgeber may be a prescription of light, designed to shift the circadian clock while not exceeding ten hours of continuous darkness at any point. In an embodiment, a zeitgeber may include a pulse of activity and a schedule of light exposure with no budgets. In an embodiment, a zeitgeber may include a pulse of activity and a schedule of light exposure with budgets imposed. In an embodiment, K(t) might be computed from data collected from a wearable device.

In an embodiment, the target trajectory y(t) when a time zone has been crossed might be the individual's ideal wake time in a target limit state of y(t) where the cost value corresponds to the number of hours of the time zones the individual crosses. In an embodiment, y(t) may be defined by the individual's ideal wake time when no time zone is crossed. In an embodiment, the ideal wake time corresponds to the time of day when awakening is predicted to occur when y(t) is coupled to a sleep model. In an embodiment, y(t) may be a limit state where peak fatigue (or core body temperature minimum) occurs outside of working hours when an individual moves to work on a night shift. In an embodiment, y(t) may be at a time that peak alertness happens during the night, while allowing sleep during the day where an individual has an erratic lighting schedule (e.g., one who works in a different time zone from where they live).

In one embodiment, a global cost is calculated after having selected a zeitgeber K₀ and integrated x(t) over a time range [tᵢ, tⱼ]; where the global cost is defined as in Equation 1, where C_{A}, C_{B} . . . C_{N} each represent different possible criteria of interest, calculated from the circadian trajectory x(t), the zeitgeber K(t) and any derivative quantities, such as W(t) and A(t), while X, Y, and Z are non-negative weighting constants.

$$C = {XC}_{A} + {YC}_{B} … + {ZC}_{N} \qquad ( Eqn . l )$$

In one embodiment, some subset of C_{A}, C_{B}, . . . , C_{N} may be set to a constant value, such as “1”. It should be noted here that each C_{A}, C_{B}, . . . C_{N} need not be equal in value.

In an embodiment, C_{A}, C_{B}, . . . , C_{N} are considered according to a defined hierarchy in evaluating a zeitgeber; where each C_{A}, C_{B}, . . . , C_{N} has a convergence threshold value, and if any cost is above that convergence threshold, all others lower in the hierarchy are set to a maximum constant value.

Assuming, that C_{A} is the higher than C_{N} in hierarchy, the following example is provided, wherein C_{A} is equal to a phase cost value, Cₚₕₐₛₑ, to capture a distance between a circadian trajectory x(t) and the target limit cycle y(t). If x(t) is approximately equal to the y(t), then the zeitgeber K(t) is deemed to have been found. However, if x(t) does not approximately equal y(t), a C_{Ai} is calculated until the cost between x(t) and y(t) falls below some defined threshold for C_{A}. Once this threshold criterion is met for C_{A}, each subsequent, C_{N}, in hierarchy turn, is then allowed to decrease as new zeitgebers K(t) are sampled until all threshold criteria are met, or some other convergence criterion, such as maximum number of iterations, is reached. In an embodiment, cost is at least one taken from the list comprising phase cost, feasibility cost, sleep duration cost, alertness cost, and/or social jet lag cost.

In an embodiment, the hierarchy of cost in the case where a time zone is crossed is (from highest to lowest) phase cost, feasibility cost, sleep duration cost, alertness cost. In an embodiment, the hierarchy of cost in the case of shift work is phase cost, sleep duration cost, alertness cost (during shift hours), feasibility cost. In an embodiment, the hierarchy of cost in the case where sleep is shifting to a preferred time is phase cost, feasibility cost, sleep duration cost, social jet lag cost, alertness cost.

In another embodiment, a global cost is calculated after having selected a zeitgeber K₀ and integrated x(t) over a time range [tᵢ, tⱼ] where the global cost is defined as in Equation 2, with C_{A}, C_{B} . . . C_{N} each representing different possible criteria of interest, calculated from a circadian trajectory x(t), a zeitgeber K(t), and any derivative quantities, such as W(t) and A(t), while X, Y, and Z are non-negative weighting constants.

$$C = {XC}_{A} + {YC}_{B} … + {ZC}_{N} \qquad ( Eqn . 2 )$$

However, no hierarchy of costs are applied, and the global cost simply reflects the raw sum of the quantities of interest. In one embodiment, cost is at least one taken from the list of phase cost, feasibility cost, sleep duration, alertness, social jetlag cost.

In another embodiment, a global or total cost is used to evaluate a zeitgeber, at time t, as in Equation 3.

$$C = {C}_{A} \times {C}_{B} \times … {C}_{N} \qquad ( Eqn . 3 )$$

In an embodiment, C_{A}, C_{B}, . . . C_{N} are considered in prescribing a zeitgeber; where each C_{A}, C_{B}, . . . C_{N} has a minimum and maximum value.

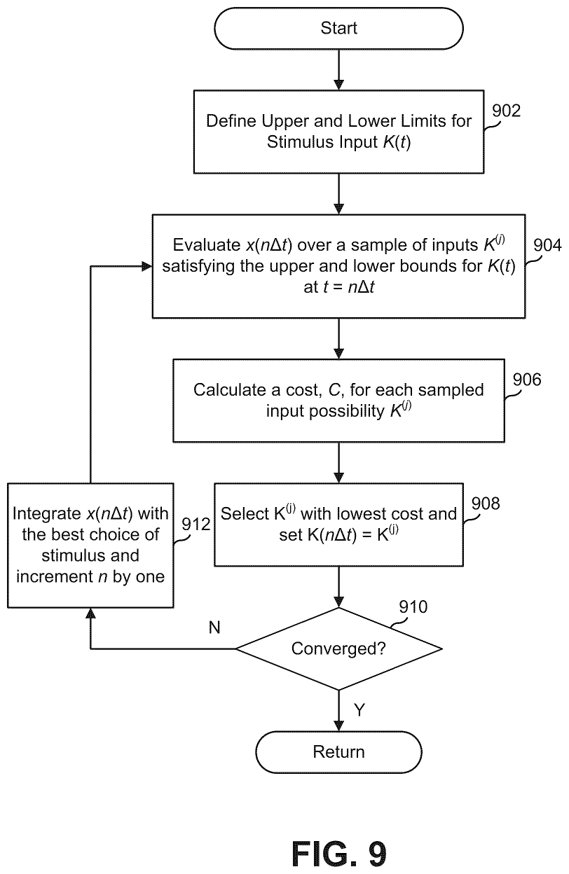

FIG. 9 is a flowchart of a method for computing a stimulus time series, K(t), with constraints. As shown, the method comprises steps 902, 904, 906, 908, 910, and 912. A vector defining a zeitgeber event at [t, tᵢ] is provided and the method comprises providing lower and upper bounds for K(t) (step 902), using a sampling method, having the components of K(t), sample within the lower and upper bounds prescribed for K(t) (904), calculating the cost from the circadian trajectory and zeitgeber, C=(x(t), K(t)) (step 906), interpolating sample points to identify possible locations of minima for C (step 908), resampling K(t) in identified possible minima locations at a denser resolution and repeating the interpolation step (912). In an embodiment, the vector defining a zeitgeber may include a duration of light, a duration of dark, and/or a timing of a zeitgeber event. In an embodiment, the upper and lower bounds of K(t) are provided as follows: for light and dark duration, the bounds are set to be the minimum and maximum allowable time spent in either dark or light; however, the first and last light and dark duration have no lower bounds; for activities specified once every N hours, such as caffeine and exercise, the lower and upper bounds are set to be k×N and (k+1)×N; where k is the specified activity.

In an embodiment, the method to determine K(t) is repeated until a defined convergence limit is reached or a maximum number of iterations is met. In an embodiment, the sampling method is Latin hypercube sampling. In an embodiment, sample points are interpolated as polynomials to identify rough locations of minima. In an embodiment, sample points are interpolated using more sophisticated curve fitting functions, such as an artificial neural net, to identify rough locations of minima.

The steps shown in FIG. 9 might be performed by a computer appropriately programmed and provided access to electronically readable representations of input values.

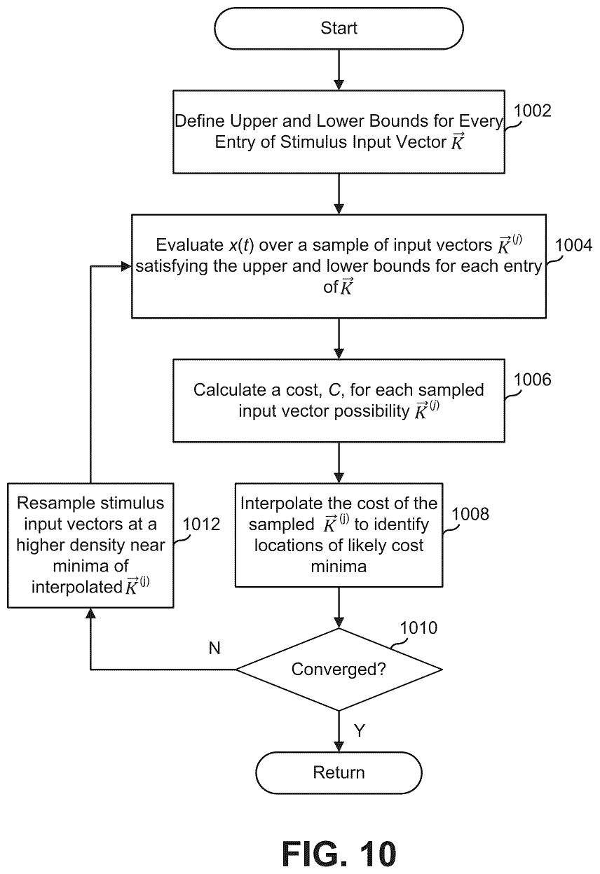

FIG. 10 is a flowchart of a method for computing a stimulus time series of input vectors, \vec{K}(t). The method might comprise steps 1002, 1004, 1006, 1008, 1010, and 1012. The steps shown in FIG. 10 might be performed by a computer appropriately programmed and provided access to electronically readable representations of input values. For example, the method might comprise determining a K(t) by calculating components of x₀, sampling from a range of possible zeitgebers K⁽¹⁾, K⁽²⁾, . . . K^{(N)} for a single time step, where each K corresponds to a different zeitgeber presentation, such as light of a certain quality, or the presence/absence of an activity, calculating a circadian trajectory x(t) for that time step for all K⁽ⁱ⁾, calculating a cost value C⁽ⁱ⁾ of each K⁽ⁱ⁾ and choosing the K⁽ⁱ⁾ with the lowest cost, setting K(t₀) to be the chosen zeitgeber values with lowest cost, and repeating the process for remaining time steps.

There might be defined upper and lower bounds for each component of the stimulus input vectors to reduce the search space. The circadian trajectory x(t) can be evaluated over a sample of input vectors that satisfy the upper and lower bounds for each component. A cost might be computed for each sampled input vector, the cost interpolated, and if not converged, resampled at a higher density near minima of the interpolated vectors.

As will be appreciated by one skilled in the art, aspects of the methods and/or apparatus described herein may be embodied as a system, method, or computer product. Accordingly, aspects thereof may take the form of an entirely hardware embodiment, entirely software embodiment (including firmware, resident software, micro-code, etc.) or an embodiment combining software and hardware aspects. Further aspects may take the form of a computer program embodied in one or more readable medium having computer readable program code/instructions thereon. Program code embodied on computer-readable medium may be transmitted using any appropriate medium, including but not limited to wireless, wireline, optical fiber cable, RF, etc., or any suitable combination of the foregoing. The computer code may be executed entirely on a user's computer, partly on the user's computer, as a standalone software package, a cloud service, partly on the user's computer and partly on a remote computer or entirely on a remote computer, remote or cloud-based server.

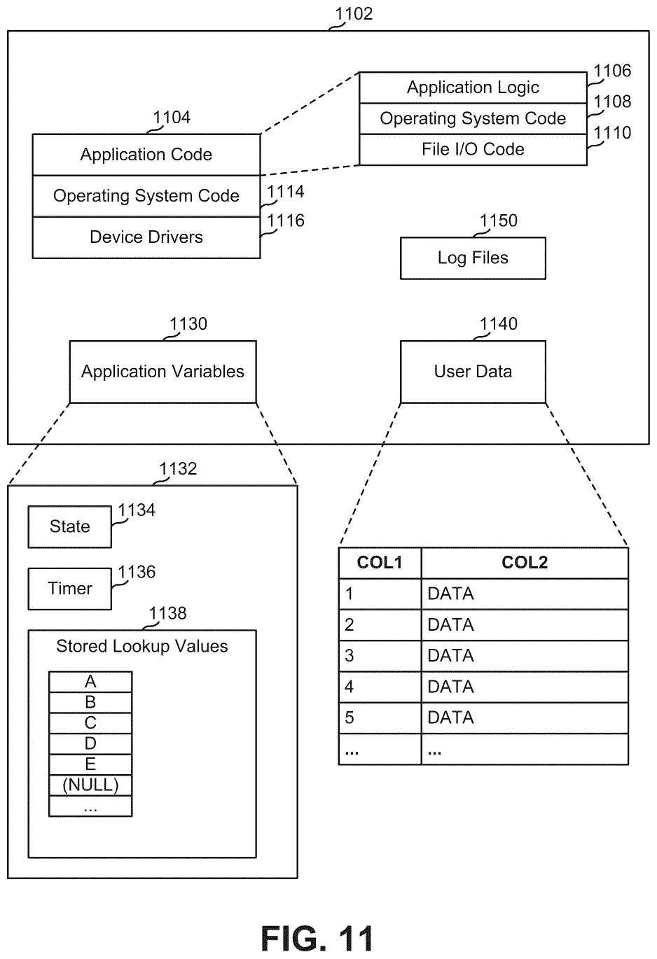

FIG. 11 is a simplified functional block diagram of a storage device 1102 having an application that can be accessed and executed by a processor in a computer system as might be part of embodiments of a circadian entrainment system and/or method and/or a computer system that optimizes circadian entrainment, or at least improves it, by considering cost functions and possibly minimizing or reducing cost function values. FIG. 11 also illustrates an example of memory elements that might be used by a processor to implement elements of the embodiments described herein. In some embodiments, the data structures are used by various components and tools, some of which are described in more detail herein. The data structures and program code used to operate on the data structures may be provided and/or carried by a transitory computer readable medium, e.g., a transmission medium such as in the form of a signal transmitted over a network. For example, where a functional block is referenced, it might be implemented as program code stored in memory. The application can be one or more of the applications described herein, running on servers, clients or other platforms or devices and might represent memory of one of the clients and/or servers illustrated elsewhere.

Storage device 1102 can be one or more memory device that can be accessed by a processor and storage device 1102 can have stored thereon application code 1104 that can be one or more processor readable instructions, in the form of write-only memory and/or writable memory. Application code 1104 can include application logic 1106, library functions 1108, and file I/O functions code 1110 associated with the application. The memory elements of FIG. 11 might be used for a server or computer that interfaces with a user, generates data, and/or manages other aspects of a process described herein. In addition to application code 1104, storage device 1102 might also contain operating system code 1114 and device drivers 1116.

Storage device 1102 can also include storage for application variables 1130 that can include one or more storage locations configured to receive variables 1132. Application variables 1130 can include variables that are generated by the application or otherwise local to the application, such as state variables 1134, timers 1136, and/or stored lookup values 1138. Application variables 1130 can be generated, for example, from data retrieved from an external source, such as a user or an external device or application. A processor can execute application code 1104 to generate application variables 1130 provided to storage device 1102. Application variables 1130 might include operational details needed to perform the functions described herein.

Storage device 1102 can include storage for databases and other data described herein. One or more memory locations can be configured to store user data 1140, which might include data sourced by an external source, such as a user or an external device. User data 1140 can include, for example, records being passed between servers prior to being transmitted or after being received. Other data might also be supplied.

Storage device 1102 can also include log files 1150 having one or more storage locations configured to store results of the application or inputs provided to the application. For example, log files 1150 can be configured to store a history of actions, alerts, error messages, and the like.

According to some embodiments, the techniques described herein are implemented by one or more generalized computing systems programmed to perform the techniques pursuant to program instructions in firmware, memory, other storage, or a combination. Special-purpose computing devices may be used, such as desktop computer systems, portable computer systems, handheld devices, networking devices or any other device that incorporates hard-wired and/or program logic to implement the techniques.

One embodiment might include a carrier medium carrying data that includes data having been processed by the methods described herein. The carrier medium can comprise any medium suitable for carrying the data, including a storage medium, e.g., solid-state memory, an optical disk or a magnetic disk, or a transient medium, e.g., a signal carrying the data such as a signal transmitted over a network, a digital signal, a radio frequency signal, an acoustic signal, an optical signal or an electrical signal.

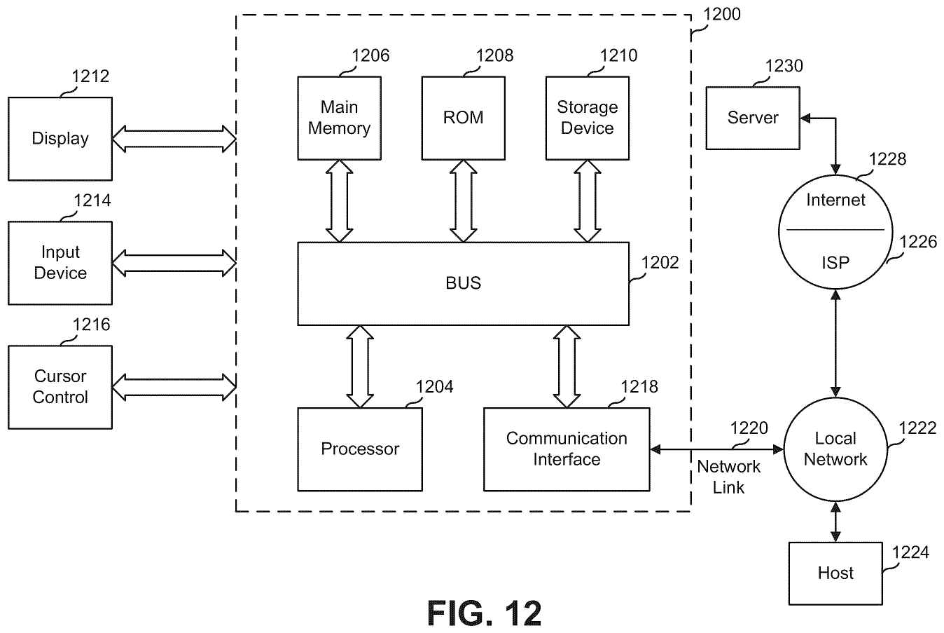

FIG. 12 is a block diagram that illustrates a computer system 1200 upon which the computer systems of the systems described herein and/or data structures shown in FIG. 11 may be implemented. Computer system 1200 includes a bus 1202 or other communication mechanism for communicating information, and a processor 1204 coupled with bus 1202 for processing information. Processor 1204 may be, for example, a general-purpose microprocessor.

Computer system 1200 also includes a main memory 1206, such as a random-access memory (RAM) or other dynamic storage device, coupled to bus 1202 for storing information and instructions to be executed by processor 1204. Main memory 1206 may also be used for storing temporary variables or other intermediate information during execution of instructions to be executed by processor 1204. Such instructions, when stored in non-transitory storage media accessible to processor 1204, render computer system 1200 into a special-purpose machine that is customized to perform the operations specified in the instructions.

Computer system 1200 further includes a read only memory (ROM) 1208 or other static storage device coupled to bus 1202 for storing static information and instructions for processor 1204. A storage device 1210, such as a magnetic disk or optical disk, is provided and coupled to bus 1202 for storing information and instructions.

Computer system 1200 may be coupled via bus 1202 to a display 1212, such as a computer monitor, for displaying information to a computer user. An input device 1214, including alphanumeric and other keys, is coupled to bus 1202 for communicating information and command selections to processor 1204. Another type of user input device is a cursor control 1216, such as a mouse, a trackball, or cursor direction keys for communicating direction information and command selections to processor 1204 and for controlling cursor movement on display 1212. This input device typically has two degrees of freedom in two axes, a first axis (e.g., x) and a second axis (e.g., y), that allows the device to specify positions in a plane.

Computer system 1200 may implement the techniques described herein using customized hard-wired logic, one or more ASICs or FPGAs, firmware, and/or program logic which in combination with the computer system causes or programs computer system 1200 to be a special-purpose machine. According to one embodiment, the techniques herein are performed by computer system 1200 in response to processor 1204 executing one or more sequences of one or more instructions contained in main memory 1206. Such instructions may be read into main memory 1206 from another storage medium, such as storage device 1210. Execution of the sequences of instructions contained in main memory 1206 causes processor 1204 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may include non-volatile media and/or volatile media. Non-volatile media includes, for example, optical or magnetic disks, such as storage device 1210. Volatile media includes dynamic memory, such as main memory 1206. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, an EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire, and fiber optics, including the wires that include bus 1202. Transmission media can also take the form of acoustic or light waves, such as those generated during radio-wave and infra-red data communications.

Various forms of media may be involved in carrying one or more sequences of one or more instructions to processor 1204 for execution. For example, the instructions may initially be carried on a magnetic disk or solid-state drive of a remote computer. The remote computer can load the instructions into its dynamic memory and send the instructions over a network connection. A modem or network interface local to computer system 1200 can receive the data. Bus 1202 carries the data to main memory 1206, from which processor 1204 retrieves and executes the instructions. The instructions received by main memory 1206 may optionally be stored on storage device 1210 either before or after execution by processor 1204.

Computer system 1200 also includes a communication interface 1218 coupled to bus 1202. Communication interface 1218 provides a two-way data communication coupling to a network link 1220 that is connected to a local network 1222. For example, communication interface 1218 may be a network card, a modem, a cable modem, or a satellite modem to provide a data communication connection to a corresponding type of telephone line or communications line. Wireless links may also be implemented. In any such implementation, communication interface 1218 sends and receives electrical, electromagnetic, or optical signals that carry digital data streams representing various types of information.

Network link 1220 typically provides data communication through one or more networks to other data devices. For example, network link 1220 may provide a connection through local network 1222 to a host computer 1224 or to data equipment operated by an Internet Service Provider (ISP) 1226. ISP 1226 in turn provides data communication services through the world-wide packet data communication network now commonly referred to as the “Internet” 1228. Local network 1222 and Internet 1228 both use electrical, electromagnetic, or optical signals that carry digital data streams. The signals through the various networks and the signals on network link 1220 and through communication interface 1218, which carry the digital data to and from computer system 1200, are example forms of transmission media.

Computer system 1200 can send messages and receive data, including program code, through the network(s), network link 1220, and communication interface 1218. In the Internet example, a server 1230 might transmit a requested code for an application program through the Internet 1228, ISP 1226, local network 1222, and communication interface 1218. The received code may be executed by processor 1204 as it is received, and/or stored in storage device 1210, or other non-volatile storage for later execution.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory. The code may also be provided carried by a transitory computer readable medium e.g., a transmission medium such as in the form of a signal transmitted over a network.

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., may be either A or B or C, or any nonempty subset of the set of A and B and C. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

The use of examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

Further embodiments can be envisioned to one of ordinary skill in the art after reading this disclosure. In other embodiments, combinations or sub-combinations of the above-disclosed invention can be advantageously made. The example arrangements of components are shown for purposes of illustration and combinations, additions, re-arrangements, and the like are contemplated in alternative embodiments of the present invention. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

All references, including publications, patent applications, and patents, cited herein are hereby incorporated by reference to the same extent as if each reference were individually and specifically indicated to be incorporated by reference and were set forth in its entirety herein.

What is claimed is:

1. A method for determining a zeitgeber, K(t), for use in a circadian entrainment system to entrain an individual's circadian clock from a current circadian trajectory, x(t), to or towards a target circadian trajectory, y(t), that minimizes or reduces a cost function C=C(x(t), y(t), K(t)), the method comprising:
(a) identifying an initial circadian state, x₀;
(b) identifying the target circadian trajectory y(t);
(c) providing an initial zeitgeber, K₀(t), and setting the zeitgeber K(t)=K₀(t);
(d) simulating the current circadian trajectory x(t) in response to the initial zeitgeber K₀(t);
(e) computing a global cost function, C(x(t), y(t), K₀(t)), based on the simulation;
(f) determining whether the global cost function meets a set of convergence criteria;
(g) if the global cost function does not meet the set of convergence criteria, updating the zeitgeber K(t) based on the global cost function;
(h) updating the global cost function based on the updated zeitgeber K(t);
(i) iterating steps (g) and (h) until the global cost function meets the set of convergence criteria; and
(j) providing the updated zeitgeber K(t) as the zeitgeber for use in the circadian entrainment system to entrain the individual's circadian clock.

2. The method of claim 1, wherein providing the initial zeitgeber, K₀(t), comprises providing a zeitgeber for a point in time or providing a zeitgeber for a time range.

3. The method of claim 1, wherein the zeitgeber for use in the circadian entrainment system comprises one or more of a duration of light, a duration of dark, and/or a timing of a zeitgeber event.

4. The method of claim 1, wherein simulating the current circadian trajectory x(t) in response to the initial zeitgeber K₀(t) comprises:
(a) receiving at least one dataset;
(b) developing at least one zeitgeber history, using the dataset;
(c) providing the dataset to a network of coupled oscillators, represented as nodes connected by edges, over a time period [tᵢ, tⱼ]; and
(d) using an output of the network of coupled oscillators for determining the global cost function.

5. The method of claim 4, wherein parameters of the coupled oscillators are tuned using an autoencoder neural network, and/or wherein the network of coupled oscillators is modeled as a reduction of a coupled phase oscillator network.

6. The method of claim 4, wherein the dataset includes data taken from one or more of an actigraphy dataset, a heart rate dataset, a light dataset, and/or a temperature dataset, and/or wherein the dataset is provided by a wearable device.

7. The method of claim 4, wherein at least a first x(t) derived from a first dataset and a second x(t) is derived from a second dataset, with the first x(t) and the second x(t) weighted by confidence in a quality of data, and/or wherein the first dataset and the second dataset are combined using a Kalman filter.

8. The method of claim 4, wherein updating the zeitgeber K(t) based on the global cost function comprises:
providing a lower bound for K(t);
providing an upper bound for K(t);
sampling components of K(t) within the lower bound and the upper bound, to derive a set of sampled components;
calculating the global cost function using the set of sampled components, the current circadian trajectory x(t), the target circadian trajectory y(t), and the zeitgeber K(t);
interpolating a set of cost values using the sampled components to identify possible locations of minima of the global cost function;
updating K(t) in identified minima locations of the possible locations at a denser resolution; and
repeating the interpolating using the denser resolution.

9. The method of claim 8, wherein the lower bound and the upper bound are set to be a minimum allowable time and a maximum allowable time, respectively, spent in either dark or light, with a first light duration, a first dark duration, a last light duration, and a last dark duration having no lower bounds, and wherein, for a specified activity, k, specified once every N hours, the lower bound is k*N and the upper bound is (k+1)*N.

10. The method of claim 8, wherein sampling components of K(t) is done using Latin hypercube sampling, and/or wherein sampled cost values are interpolated with polynomials, trigonometry functions, or neural networks.

11. The method of claim 1, wherein determining the zeitgeber comprises:
(a) calculating components of x₀;
(b) sampling from a range of possible zeitgeber choices K⁽ⁱ⁾, K^{(l)}, . . . K^{(N)}, where each zeitgeber choice K⁽ⁱ⁾ represents a different choice of stimulus for a single time step;
(c) simulating x(t) over a single time step to get x₁ for each zeitgeber choice K⁽ⁱ⁾;
(d) calculating a cost for all zeitgeber choices K⁽ⁱ⁾;
(e) choosing the zeitgeber choice K⁽ⁱ⁾ with a lowest cost, Cⁱ, as a chosen zeitgeber;
(f) setting K(t₀) equal to the chosen zeitgeber; and
(g) repeating steps (c) through (f) for xₙ.

12. The method of claim 1, wherein C(x(t), y(t), K(t))=XC_{A}+YC_{B}+ . . . +ZC_{N}, with C_{A}, C_{B}, . . . , C_{N} each representing different possible criteria of interest and X, Y, Z each representing weightings of these costs, and further wherein C_{A}, C_{B}, . . . , C_{N} are considered according to a defined hierarchy in evaluating a zeitgeber.

13. The method of claim 12, wherein each of C_{A}, C_{B}, . . . , C_{N} is based on one or more of a phase cost, a feasibility cost, a sleep duration, an alertness value, and/or a social jetlag cost.

14. The method of claim 1, wherein the global cost function representing a cost to approximately entrain to a target circadian state is minimized when C(x(t), y(t), K(t)) is less than a threshold value of cost.

15. The method of claim 1, wherein C(x(t), y(t), K(t))=C_{A}*C_{B}* . . . *C_{N}, and wherein each C_{A}, C_{B}, . . . , C_{N} has a minimum and maximum value.

16. The method of claim 1, wherein C is based on one or more of a phase cost, a feasibility cost, a sleep duration, an alertness value, and/or a social jetlag cost.

17. A computer system for generating a zeitgeber, K(t), for use in a circadian entrainment system to entrain an individual's circadian clock from a current circadian trajectory, x(t), to or towards a target circadian trajectory, y(t), that minimizes or reduces a cost function C=C(x(t), y(t), K(t)), the computer system comprising:
at least one processor; and
a computer-readable medium storing instructions, which when executed by the at least one processor, causes the computer system to:
(a) identify an initial circadian state, x₀;
(b) identify the target circadian trajectory y(t);
(c) provide an initial zeitgeber, K₀(t), and setting the zeitgeber K(t)=K₀(t);
(d) simulate the current circadian trajectory x(t) in response to the initial zeitgeber K₀(t);
(e) compute a global cost function, C(x(t), y(t), K₀(t)), based on the simulation;
(f) determine whether the global cost function meets a set of convergence criteria;
(g) test whether the global cost function meets the set of convergence criteria;
(h) update the zeitgeber K(t) based on the global cost function upon determining that the global cost function does not meet the set of convergence criteria;
(i) update the global cost function based on the updated zeitgeber K(t);
(j) iterating steps (h) and (i) until the global cost function meets the set of convergence criteria; and
(k) providing the updated zeitgeber K(t) as the zeitgeber for use in the circadian entrainment system to entrain the individual's circadian clock.

18. The computer system of claim 17, further comprising a network of coupled oscillators represented as nodes connected by edges, wherein the computer-readable medium storing instructions further stores instructions to:
(l) receive at least one dataset;
(m) develop at least one zeitgeber history, using the dataset;
(n) provide the dataset to the network of coupled oscillators over a time period [tᵢ, tⱼ]; and
(o) use an output of the network of coupled oscillators for determining the global cost function.

19. The computer system of claim 17, wherein the computer-readable medium storing instructions further stores instructions to:
(l) provide a lower bound for K(t) and an upper bound for K(t);
(m) sample components of K(t) within the lower bound and the upper bound, to derive a set of sampled components;
(n) calculate the global cost function using the set of sampled components, the current circadian trajectory x(t), the target circadian trajectory y(t), and the zeitgeber K(t);
(o) interpolate a set of cost values using the sampled components to identify possible locations of minima of the global cost function;
(p) update K(t) in identified minima locations of the possible locations at a denser resolution; and
(q) repeating the interpolating using the denser resolution.

20. The computer system of claim 17, wherein the computer-readable medium storing instructions further stores instructions to:
(l) calculating components of x₀;
(m) sampling from a range of possible zeitgeber choices K⁽ⁱ⁾, K⁽²⁾, . . . K^{(N)}, where each zeitgeber choice K⁽ⁱ⁾ represents a different choice of stimulus for a single time step;
(n) simulating x(t) over a single time step to get x₁ for each zeitgeber choice K⁽ⁱ⁾;
(o) calculating a cost for all zeitgeber choices K⁽ⁱ⁾;
(p) choosing the zeitgeber choice K⁽ⁱ⁾ with a lowest cost, Cⁱ, as a chosen zeitgeber;
(q) setting K(t₀) equal to the chosen zeitgeber; and
(r) repeating steps (n) through (q) for xₙ.
